(19) United States
(12) Patent Application Publication
(10) Pub. No.: US 2026/0145402 A1
(43) Pub. Date: May 28, 2026

(54) CUSTOMIZABLE ORTHOTIC INSOLE MANUFACTURING USING VISCOUS THREAD PRINTING

(71) Applicant: Northeastern University, Boston, MA (US)

(72) Inventors: Jeffrey Lipton, Sharon, MA (US); Brett Alexander Emery, Boston, MA (US)

(21) Appl. No.: 19/401,797

(22) Filed: Nov. 26, 2025

Related U.S. Application Data
(60) Provisional application No. 63/725,606, filed on Nov. 27, 2024.

(51) Int. Cl. B29D 35/12; A61B 5/107; B29C 64/393; B29D 35/14; B33Y 10/00; B33Y 30/00; B33Y 50/02; B33Y 80/00

(52) U.S. Cl. CPC B29D 35/122; A61B 5/1074; A61B 5/1079; B29C 64/393; B29D 35/148; B33Y 10/00; B33Y 30/00; B33Y 50/02; B33Y 80/00

(57) ABSTRACT

A method for manufacturing a customized orthotic insole, the method including: obtaining a three-dimensional model of an individual's foot; generating a three-dimensional model of an orthotic insole based on the three-dimensional model of the individual's foot; dividing the three-dimensional model of the orthotic insole into multiple zones, each zone having specified mechanical properties; and generating printer instructions for fabricating the orthotic insole using viscous thread printing, wherein the printer instructions are configured to produce the specified mechanical properties in each zone by controlling viscous thread coiling parameters during deposition of viscous threads.

### CROSS REFERENCE TO RELATED APPLICATIONS

This application claims the benefit of priority to U.S. Provisional Application No. 63/725,606, filed on Nov. 27, 2024, the entire contents of which are hereby incorporated by reference.

### BACKGROUND

Orthotic insoles are widely used to provide support, cushioning, and correction for various foot conditions and biomechanical issues. Traditionally, custom orthotics have been manufactured through labor-intensive processes involving manual casting, molding, and assembly of multiple materials. These methods often result in long production times, high costs, and limited ability to fine-tune the mechanical properties of different regions within the orthotic.

Recent advancements in 3D printing technologies have opened new possibilities for manufacturing customized orthotic devices. However, existing 3D printing methods for orthotics typically rely on rigid materials or multi-material approaches that can lead to durability issues at material interfaces. Additionally, current techniques often lack the ability to precisely control and gradually vary the mechanical properties throughout the orthotic structure, limiting the potential for optimized biomechanical support and comfort for individual users.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a method for manufacturing a customized orthotic insole, the method comprising: obtaining a three-dimensional model of an individual's foot; generating a three-dimensional model of an orthotic insole based on the three-dimensional model of the individual's foot; dividing the three-dimensional model of the orthotic insole into multiple zones, each zone having specified mechanical properties; and generating printer instructions for fabricating the orthotic insole using viscous thread printing, wherein the printer instructions are configured to produce the specified mechanical properties in each zone by controlling viscous thread coiling parameters during deposition of viscous threads.

In some embodiments, obtaining a three-dimensional model of the individual's foot comprises capturing a shape and a plurality of contours of the individual's foot with a three-dimensional scanner.

In some embodiments, generating the three-dimensional model of the orthotic insole further comprises analyzing at least one of an anatomical landmark, a gait analysis or a pressure map of the individual.

In some embodiments, the specified mechanical property comprises at least one of stiffness, flexibility or porosity.

In some embodiments, dividing the three-dimensional model of the orthotic insole into multiple zones comprises identifying transition areas between the multiple zones.

In some embodiments, generating printing instructions comprises: generating a digital representation of the orthotic insole's three-dimensional structure including the multiple zones; and generating viscous thread coiling parameters based on the specified mechanical properties of each zone and transition areas of the orthotic insole.

In some embodiments, the printer instructions comprises at least one of extrusion rate, print head translation speed, print height, and thread diameter.

In some embodiments, generating the printing instructions comprises applying continuous property gradients corresponding to the transition areas between identified zones by gradually varying the viscous thread coiling parameters.

In some embodiments, the method further comprises determining viscous thread coiling parameters via a predictive model configured to map the viscous thread coiling parameters to the specified mechanical properties at each identified zone and transition area.

The herein disclosed subject matter is also directed to a system for manufacturing a customized orthotic insole, the system comprising: a three-dimensional scanner configured to obtain a three-dimensional model of an individual's foot; a processor configured to: generate a three-dimensional model of an orthotic insole based on the three-dimensional model of the individual's foot; divide the three-dimensional model of an orthotic insole into a plurality of zones, each zone having specified mechanical properties; and generate viscous thread printing instructions including a plurality of viscous thread coiling parameters to print an orthotic insole based on the three-dimensional model of the orthotic insole and the specified mechanical properties of each zone; a viscous thread printing device configured to deposit viscous threads based on the viscous thread printing instructions.

In some embodiments, the plurality of viscous thread coiling parameters comprises at least one of extrusion rate, print head translation speed, print height and thread diameter.

In some embodiments, the system further comprises a material database storing relationships between viscous thread coiling parameters and specified mechanical properties for at least one thermoplastic material.

In some embodiments, the processor is configured to use a predictive model to determine viscous thread coiling parameters that will produce the specified mechanical properties in each zone.

In some embodiments, wherein the processor is further configured to apply continuous property gradients between adjacent zones by gradually varying the viscous thread coiling parameters in the printing instructions.

The herein disclosed subject matter is also directed to a customized orthotic insole produced by a process comprising the steps of: obtaining a three-dimensional model of an individual's foot; generating a three-dimensional model of an orthotic insole based on the three-dimensional model of the individual's foot; dividing the three-dimensional model of the orthotic insole into multiple zones, each zone having specified mechanical properties; dividing the three-dimensional model of the orthotic insole into at least one transition area between adjacent zones; generating printer instructions for fabricating the orthotic insole using viscous thread printing, the printer instructions are configured to produce the specified mechanical properties in each zone and transition area by controlling viscous thread coiling parameters during deposition of viscous threads; and printing the customized orthotic insole via a viscous thread printing device based on the printer instructions, the customized orthotic insole comprising the plurality of viscous threads deposited in layers to form a three-dimensional structure the specified mechanical properties in a corresponding one of the multiple zones and transition area.

In some embodiments, the customized orthotic insole is fabricated from a single thermoplastic material.

In some embodiments, each of specified mechanical properties corresponding to each zone and transition area is achieved through variation of the viscous thread coiling parameters.

In some embodiments, the customized orthotic insole further comprises a high-density accumulation region of viscous threads forming at least one of a wall and a base of the orthotic insole.

In some embodiments, a top surface of the orthotic insole is ironed by the viscous thread printer during the printing.

In some embodiments, a top surface of the orthotic insole is printed with a plurality of loops formed by the viscous threads, the loops configured to receive and engage with a replaceable top surface layer having a plurality of hooks.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description. It is to be understood that both the foregoing general description and the following detailed description are exemplary and are intended to provide further explanation of the disclosed subject matter claimed.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part

FIG. 1 is a schematic representation of a system for fabricating a customized orthotic insole in accordance with embodiments of the present disclosure

FIG. 2 is a schematic planform view of a three-dimensional model of an orthotic insole divided into a plurality of zones and transition areas in accordance with embodiments of the present disclosure.

FIG. 3 is an exemplary method for fabricating a customized orthotic insole in accordance with embodiments of the present disclosure.

FIG. 4A is a top perspective view of a customized orthotic insole formed from viscous thread printing in accordance with embodiments of the present disclosure.

FIG. 4B is a side elevation view of a customized orthotic insole formed from viscous thread printing in accordance with embodiments of the present disclosure.

FIG. 4C is a top perspective view of a customized orthotic insole formed from viscous thread printing showing surface contours in accordance with embodiments of the present disclosure.

FIG. 4D is a side view of a customized orthotic insole formed from viscous thread printing showing a plurality of layers of depositing viscous threads in accordance with embodiments of the present disclosure.

FIG. 4E is a planform view of a customized orthotic insole formed from viscous thread printing in accordance with embodiments of the present disclosure.

FIG. 5 is a schematic diagram of a computing node in accordance with embodiments of the present disclosure.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

Viscous thread printing (VTP) is the process by which thin deformable threads may be deposited into coil-based foam microstructures on material extrusion three-dimensional (3D) printers. The herein disclosed systems and methods may leverage the viscous properties of thermoplastics during deposition to precisely control the coil behavior at the point of deposition by altering the relative extrusion and/or translation rate (and the relative extrusion and translations rates) and the relative print height vs thread diameter. In various embodiments, these ratios, among others, may allow various coil densities to be selectively placed to locally manifest macro-scale mechanical properties such as stiffness and porosity. These properties may also be continuously graded according to one or more prescribed parameter curves to smoothly transition from one property space to another without leaving discrete boundaries at property zone interfaces or relying on multistep assembly of multiple materials using adhesives. By extensively mapping input parameters to output mechanical properties, a predictive model for navigating the available mechanical property space can be created and utilized to grant the ability to apply inverse design to desired mechanical response.

In order to facilitate the application of the VTP process and its parameter mapping during fabrication, a bespoke software known as a slicer was developed for converting 3D meshes (STLs) into printer readable ‘.gcode’ files according to the desired VTP process parameters for each zone and the respective gradients to be applied at each adjacent mesh boundaries.

By leveraging the local property control of VTP combined with the newly realized parameter mapping, a fully customized, breathable and durable orthotic may be fabricated. For example, and without limitation, one implementation of the disclosed systems and methods may allow orthotic insole fabrication at a local pediatrist clinic, for example, within 24 hours.

By scanning an individual's (e.g., patient, subject, athlete) foot, or a portion thereof, a detailed 3D model of specific foot geometry can be used to produce a ready-for-use orthotic tailored to the pressure points of the individual's feet. In various embodiments, low stiffness regions can be placed at each pressure point, while high stiffness regions can be strategically placed for support, with continuous gradients ensuring proper integration and durability of each zone.

Additional super-high density (i.e., accumulation) regions can be applied to one or more walls and a base of the orthotic for enhanced durability. Further, accumulation regions may be additionally or alternatively integrated via small gradients into the functional internal portions of the insole to form a solid, durable foundation and perimeter for the orthotic. Moreover, in various embodiments, a top surface can also be applied either by additions or subtractions (i.e., ironing) made by the 3D printer without human intervention, or by post-processing the insole by an operator to apply an external top surface. In various embodiments, the top surface can be formed from ethylene-vinyl acetate (EVA) foam. In various embodiments, hook and loop closures may be used in conjunction with the existing VTP induced loops exposed on the surface to allow for a removable and rapidly replaceable top surface.

By applying VTP to orthotic production, fully customized orthotics with multiple property zones having durable constructions can be rapidly produced onsite using readily available machines and base materials with little to no necessary postprocessing.

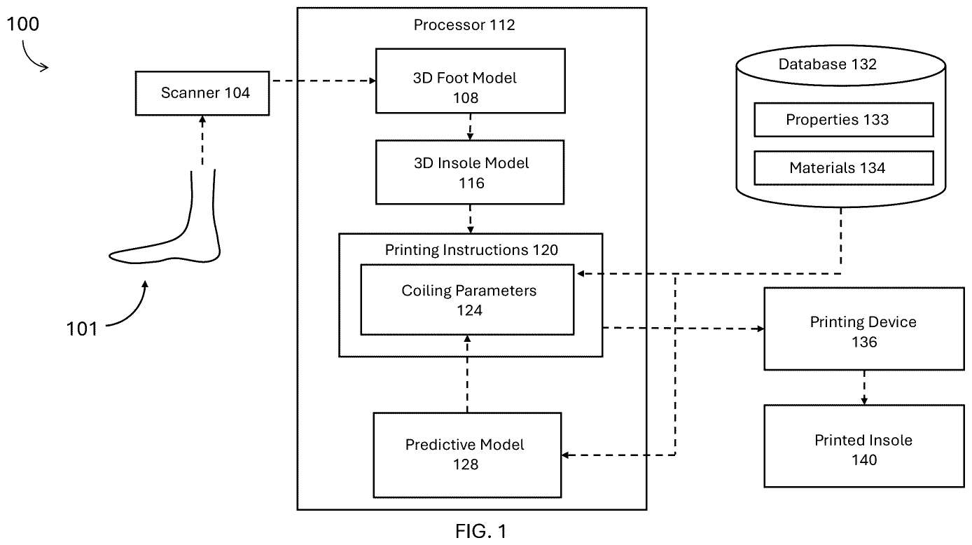

Referring now to FIG. 1, a system 100 for fabricating a customized orthotic insole is shown in schematic form. System 100 may include a scanner 104. Scanner 104 may scan an individual's foot 101 using various scanning methodologies and apparatus configurations. Scanner 104 may be handheld, tabletop, or an apparatus in which the individual inserts their foot 101. Alternatively, scanner 104 may comprise a floor-mounted scanning platform, a portable scanning unit, or a multi-directional scanning array that captures foot geometry from multiple angles simultaneously. In various embodiments, scanner 104 may include one or more electromagnetic sensors, lasers, optical sensors, ultrasound transducers, structured light projectors, photogrammetry systems, or the like in order to produce a point cloud corresponding to the geometric features of an individual's foot 101. Additionally, or alternatively, scanner 104 may employ magnetic resonance imaging (MRI), computed tomography (CT), or other volumetric imaging techniques to capture internal foot structure. In various embodiments, scanner 104 may include one or more mats or pads that map the contours of the bottom of an individual's foot 101 when stepped on. These mats or pads may incorporate pressure-sensitive elements, capacitive sensors, or piezoelectric transducers to capture both geometric and pressure distribution data. In various embodiments, scanner 104 may capture one or more of an anatomical landmark of an individual's foot 101, including but not limited to arch height, heel width, toe length, metatarsal positioning, or navicular drop measurements. In various embodiments, scanner 104 may capture one or more of a gait analysis of an individual's foot 101 during various locomotive modalities, such as walking, running, jogging, jumping or standing, among others. The gait analysis may include temporal-spatial parameters, kinematic data, kinematic measurements, or electromyographic signals. In various embodiments, scanner 104 may capture one or more of a pressure map of an individual's foot 101, such as high-pressure points associated with anatomical portions of the user's foot, or a dynamic pressure measurement of foot 101 during an aforementioned locomotive modality, among others. The pressure mapping may utilize force plates, pressure-sensitive insoles, or plantar pressure measurement systems.

In certain embodiments, scanner 104 may receive data associated with a gait analysis, anatomical landmark, pressure map or other individual data associated with the target user of the orthotic insole. For example, and without limitation, scanner 104 may be utilized in tandem with an individual's medical or orthopedic history to map one or more geometric features of the individual's foot, such as injury history, medical or mechanical conditions, or the like. The medical history may include previous surgeries, chronic conditions, biomechanical abnormalities, or therapeutic interventions. Scanner 104 may be communicatively coupled to processor 112, which in turn may receive the geometric and/or spatial data associated with the individual's foot 101 from the scanner 104, and further generate the 3D model 108 of the individual's foot. The communication between scanner 104 and processor 112 may occur through wired connections, wireless protocols, cloud-based data transfer, or direct digital interfaces. In various embodiments, the 3D foot model 108 may include contours of the sole of the individual's foot 101, or contours of the entire foot 101, such as the top of the foot, sides of the foot, and the ankle. Additionally, the 3D foot model 108 may incorporate volumetric data, surface texture information, or material property estimations of the foot tissues.

With continued reference to FIG. 1, system 100 may include a processor 112. In various embodiments, processor 112 may include one or more processors, computing devices, microcontrollers, field-programmable gateways (FPGAs), application-specific integrated circuits (ASICs), or distributed computing systems, as described below. Processor 112 may receive geometric and spatial data associated with an individual's foot 101 through various data acquisition methods and communication protocols. In certain embodiments, processor 112 may receive a previously generated 3D foot model 108 from scanner 104 or another component, such as external databases, cloud storage systems, or third-party imaging services. Processor 112 may analyze and process 3D foot model 108 to identify key anatomical landmarks of the individual, such as biological or biomechanical conditions, such as flat feet, high arches, heel and ball pressure points, and the toes of the individual. The analysis may employ machine learning algorithms, pattern recognition techniques, or biomechanical modeling software to identify these landmarks. In various embodiments, processor 112 may map static or dynamic pressure points of the 3D foot model 108 using computational fluid dynamics, finite element analysis or statistical modeling approaches. In certain embodiments, processor 112 may determine, based on the 3D foot model 108, areas of the individual's foot 101 that require support, cushioning, flexibility, rigidity, or the like through biomechanical simulation, comparative analysis with normative databases, or clinical decision support algorithms.

Processor 112 may apply biomechanical design principles to generate a 3D model of an orthotic insole 116 (interchangeably referred to as 3D insole model 116) based on the characteristics of the 3D foot model 108. The biomechanical design principles may include gait optimization algorithms, pressure redistribution calculations, or kinematic correction methodologies. Processor 112 may receive anatomical data of the individual associated with the 3D foot model 108, such as a gait analysis, medical history, injury or biomechanical conditions or the like. The anatomical data may be obtained from electronic health records, clinical assessments, wearable sensors, or patient-reported outcome measures. The 3D insole model 116 generated by processor 112 may address the specific needs of the individual based on the 3D foot model 108, through customized geometry, material property specifications, or therapeutic feature integration.

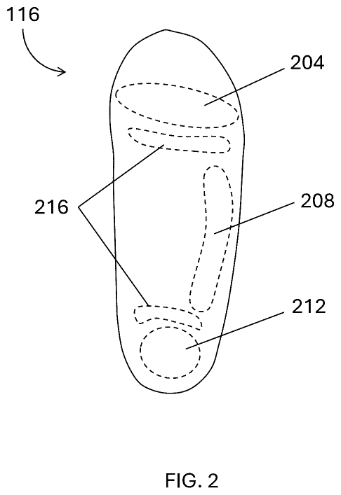

Processor 112 may divide the 3D insole model 116 into a plurality of zones (as shown in FIG. 2) using automated segmentation algorithms, manual delineation tools, or hybrid approaches combining computational and clinical expertise. Each zone (204, 208, 212, 220) may correspond to geometric features of the insole that contact an individual's foot 101. For example, and without limitation, the multiple zones may correspond to the heel, arch, instep, outstep, ball of the foot, toes, among others. Alternatively, the zones may be defined by functional regions such as weight-bearing areas, propulsive zones, or stabilization regions. In various embodiments, the multiple zones may correspond to portions of the 3D insole model 116 having specified mechanical properties such as elastic modulus, damping characteristics, or energy return capabilities. Processor 112 may incorporate anatomical landmarks identified in 3D foot model 108, pressure distribution data or biomechanical analysis of the individual's gait to divide the 3D insole model 116 into a plurality of zones. The incorporation may utilize weighted algorithms, multi-criteria decision analysis, or optimization techniques to balance competing design objectives. Accordingly, each zone of the multiple zones may allow for customization of the orthotic insole to address specific needs of different areas of the individual's foot. For example, and without limitation, zones corresponding to high-pressure areas may be designed with greater cushioning or support, while zones requiring more stability may be assigned higher stiffness specified mechanical properties. The design may incorporate shock absorption materials, energy-dissipating structures, or load-distributing geometries. For example, and without limitation, a zone (such as zone 212) which corresponds to the individual's heel may provide higher cushioning through increased porosity, softer material properties, or impact-absorbing microstructures, whereas a zone (such as zone 204) corresponding to the ball of the user's foot may provide higher flexibility to allow relative deforming of the orthotic insole during walking or running through reduced cross-linking density, optimized coil spacing, or directional material orientation.

Processor 112 may further identify one or more transition areas (such as transition area 216 in FIG. 2) disposed between the identified zones (204, 208, 212, etc.) through gradient analysis, boundary optimization, or continuity algorithms. Transition areas provide gradually varying mechanical properties associated with the adjacent zones, such that a single printing process can print the plurality of zones through the transitional areas 216 continuously without discrete interfaces or material discontinuities. Transition areas 216 may include target mechanical properties such as stiffness, flexibility, or porosity, which can be achieved through controlled variation of the viscous thread printing parameters during fabrication. The mechanical properties may transition linearly, exponentially, or according to custom mathematical functions that optimize biomechanical performance. Processor 112 may automatically divide the 3D insole model 116 based on intended target mechanical properties, biomechanical target characteristics or individual preferences using machine learning models, expert systems, or user-interactive design interfaces.

With continued reference to FIG. 1, processor 112 may further generate viscous thread printing (VTP) instructions 120 through automated code generation, template-based programming, or custom algorithm development. VTP instructions 120 may be automatically generated to print an orthotic insole based on the 3D insole model 116 having specified mechanical properties 133 associated with a material 134. The generation may employ slicing algorithms, toolpath optimization, or process parameter mapping to convert the 3D model into machine-readable instructions. VTP instructions 120 may include viscous thread coiling parameters 124 such as coil density, pitch, diameter, or spatial orientation. In various embodiments, VTP instructions 120 may include one or more of extrusion rate, print head translation speed, print height, and thread diameter to achieve desired viscous thread coiling parameters 124. Additionally, the instructions may specify nozzle temperature, bed temperature, cooling rates, or environmental controls. In various embodiments, viscous thread coiling parameters 124 and/or VTP instructions 120 may include ironing instructions, in which one or more extrusion heads of the printing device 140 smooths portions of the depositing viscous threads through controlled contact pressure, temperature regulation, or surface finishing techniques. In certain embodiments, viscous thread coiling parameters 124 may be adjusted zonally to achieve target specified mechanical properties 133 in said zone through parameter interpolation, lookup tables, or real-time feedback control. Further, processor 112 may apply continuous property gradients between adjacent zones (e.g., transition area 216) by gradually varying the viscous thread coiling parameters 124 in the VTP instructions 120 using mathematical functions, spline interpolation, or gradient descent optimization. In certain embodiments, VTP instructions 120 may be automatically transmitted to one or more printing devices 136 to print orthotic insole 140 that has the specified mechanical properties 133 in each zone through direct communication protocols, network transmission, or cloud-based manufacturing systems.

With continued reference to FIG. 1, processor 112 may generate VTP instructions 120 via a predictive model 128 that employs machine learning algorithms, neural networks, regression analysis, or other computational modeling techniques. In various embodiments, predictive model 128 may determine, based on stored relationships between viscous thread coiling parameters 124 and resulting specified mechanical properties 133 for at least one material 134, VTP instructions 120 which will, upon deposition of viscous threads, affect the viscous thread coiling parameters 124 that will achieve the specified mechanical properties 133. The determination may utilize inverse modeling, optimization algorithms, or iterative refinement processes. Predictive model 128 may be formed by extensively mapping input parameters to output mechanical properties through experimental validation, computational simulation, or hybrid modeling approaches, wherein predictive model 128 may navigate the mechanical property space, granting the ability to apply inverse design to desired mechanical response. The navigation may employ multi-objective optimization, Pareto frontier analysis, or constraint satisfaction techniques.

Processor 112 and predictive model 128 may be communicatively coupled to database 132 that stores the relationships between viscous thread coiling parameters 124 and resulting specified mechanical properties 133 for the at least one material 134 through relational databases, NoSQL systems, distributed storage architectures, or cloud-based data repositories. For example, and without limitation, VTP instructions 120 may specify motions and operational characteristics of printing device 136 to deposit coils of viscous threads having viscous thread coiling parameters 124 that affect the specified mechanical properties 133 for a given material 134 in each identified zone (204, 208, 212) and transition areas 216 with which the orthotic insole 140 is printed. The specifications may include G-code commands, proprietary machine instructions, or standardized manufacturing protocols. In various embodiments, the material 134 may be one or more thermoplastic materials suitable for viscous thread printing, such as polylactic acid (PLA), acrylonitrile butadiene styrene (ABS), thermoplastic polyurethane (TPU), or specialized biocompatible polymers. Predictive model 128 may further gradually apply continuous property gradients between adjacent zones (204, 208, 212), such as in the transition areas 216, by gradually varying the viscous thread coiling parameters 124 in the printer instructions 120 through parametric equations, interpolation functions, or adaptive control algorithms.

With continued reference to FIG. 1, system 100 may include a viscous thread printing device 136 (interchangeably called printer, VTP device, or printing device 136) that may comprise various additive manufacturing technologies and apparatus configurations. Printing device 136 may print an orthotic insole 140 based on the 3D insole model 116, having specified mechanical properties 133, utilizing the VTP instructions 120 through layer-by-layer deposition, continuous extrusion, or hybrid manufacturing processes. Printing device 136 may be a tabletop 3D printer, industrial-scale manufacturing system, or portable fabrication unit. Alternatively, printing device 136 may comprise a multi-head extrusion system, robotic manufacturing cell, or automated production line. Printing device 136 may be one or more 3D printers operatively and communicatively coupled to one another to print orthotic insole 140 continuously, simultaneously or in stages through coordinated manufacturing protocols, distributed processing systems, or synchronized production workflows.

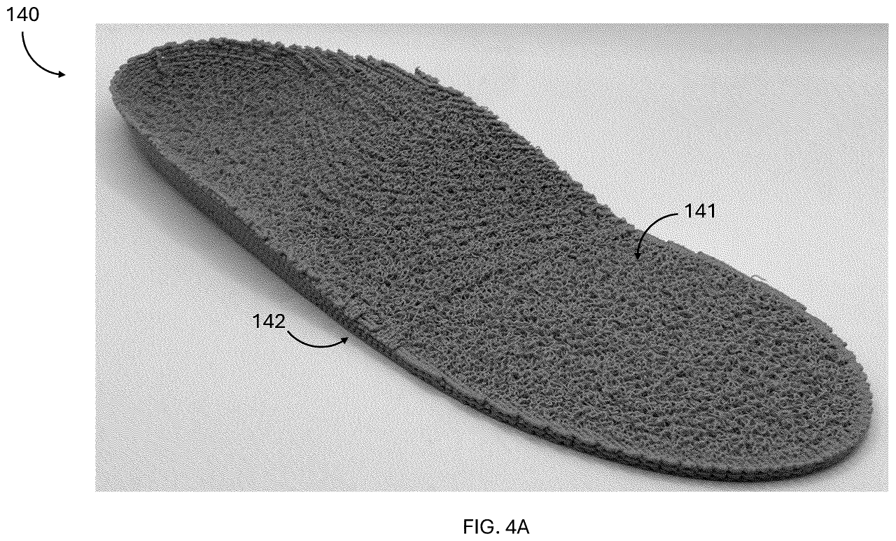

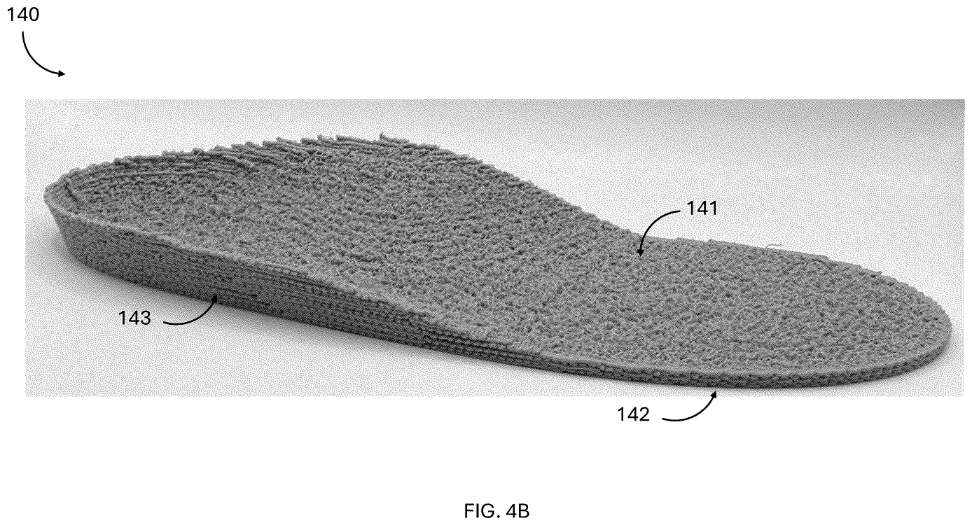

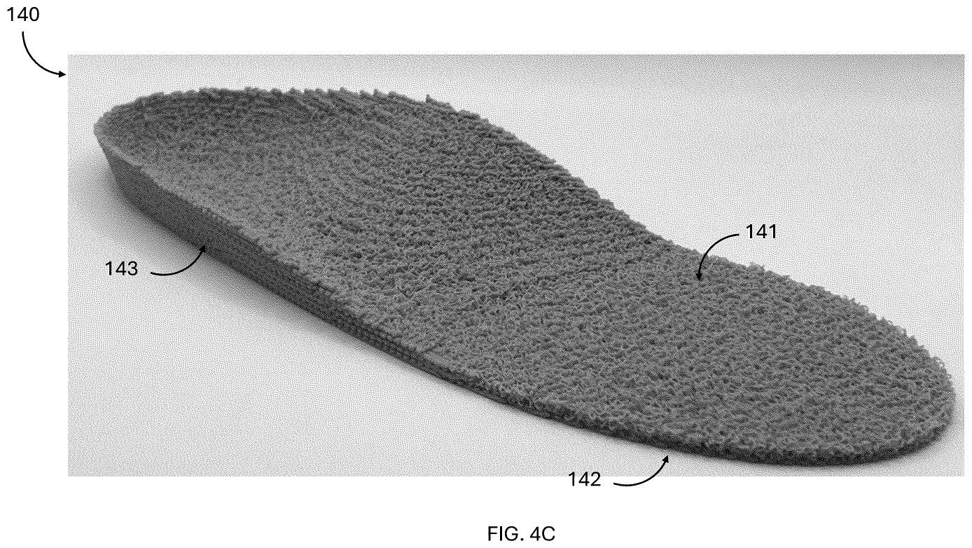

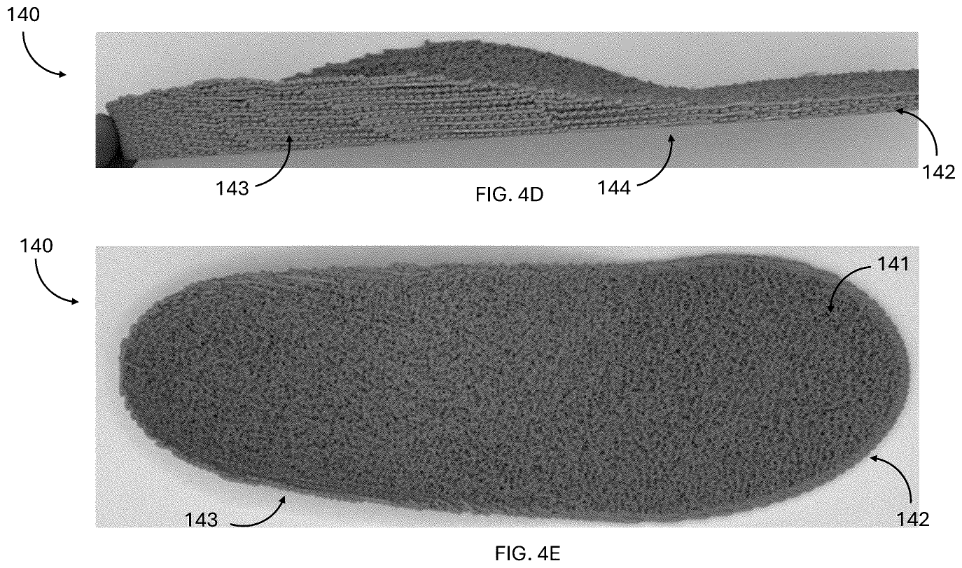

As described herein, printing device 136 may print orthotic insole 140, as shown in FIGS. 4A-4E. Orthotic insole 140 may be a customized orthotic insole configured for use by an individual based on the 3D foot model 108 and target mechanical properties in respective zones thereof. As shown in FIGS. 4A-4E, orthotic insole 140 may be a three-dimensional structure fabricated using viscous thread printing. The three-dimensional structure comprises multiple zones having different specified mechanical properties as described herein. The specified mechanical properties 133 may be produced in each zone by varying the viscous thread coiling parameters 124 during deposition of the viscous threads forming the three-dimensional structure. Orthotic insole 140 may include continuous property gradients between adjacent zones (e.g., in the transition zones 216), produced by gradually varying the viscous thread coiling parameters during fabrication. In various embodiments, specified mechanical properties 133 may include one or more of stiffness, density, porosity and flexibility, among others. In certain n embodiments, orthotic insole 140 may be fabricated from a single thermoplastic material, with distinct mechanical properties 133 in each zone 204, 208, 212 achieved solely through variation of the viscous thread coiling parameters 124. In certain embodiments, orthotic insole 140 may include high-density accumulation regions forming at least one of a wall (143 shown in FIGS. 4A-4E), base (144 shown in FIGS. 4A-4E), top surface (141 shown in FIGS. 4A-4E), edge (142 shown in FIGS. 4A-4E), or interstitial portion of the orthotic insole. Further, each accumulation region may be integrated via small gradients into the functional internal portions of the insole to form a solid, durable foundation and perimeter of the orthotic. Moreover, orthotic insole 140 may include a top surface 141. Top surface 141 may be applied after fabrication by viscous thread printing or formed continuously and integrally by viscous thread printing. In various embodiments, top surface 141 may be formed by additions or subtractions performed by the viscous thread printing device 136. For example, and without limitation, top surface 141 may be formed by ironing the topmost layer of viscous threads, where the relatively warm extrusion head of the printing device 136 contacts the viscous threads, smoothing and flattening said topmost layer. In another example, a distinct and separate top surface 141 may be applied to orthotic insole 140 after fabrication, in said example, deposited viscous threads of the orthotic insole 140 may be formed into loops, and a corresponding portion of hooks disposed on the distinct top surface 141 is brought into contact to matingly couple the top surface 141 to the orthotic insole 140. The distinct top surface 141 may be formed from an EVA foam and be configured for rapid removability and replaceability.

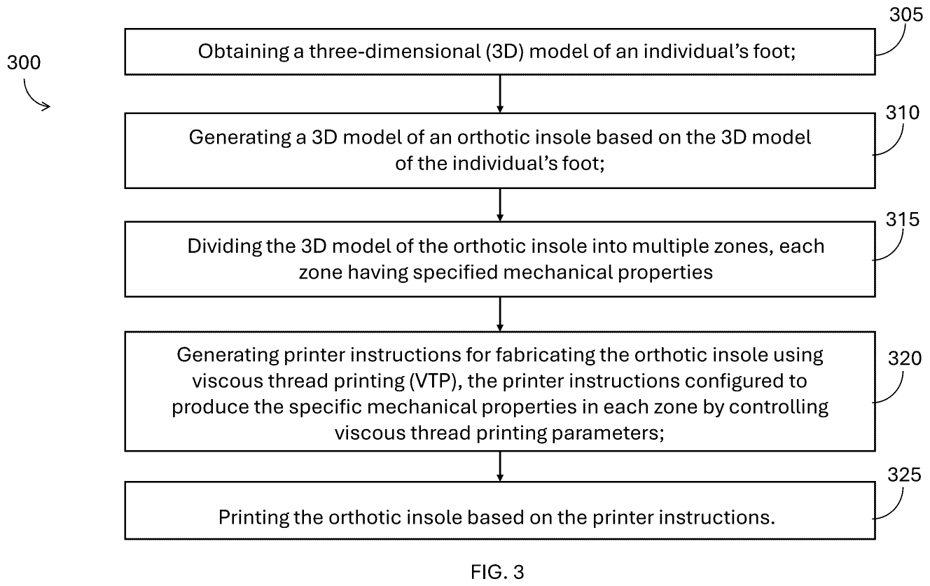

Referring now to FIG. 3, a method 300 for fabricating a customized orthotic insole is shown in flowchart form. Method 300 may include, at step 305, obtaining a three-dimensional model (3D foot model) of an individual's foot. Alternatively, step 305 may involve acquiring, capturing, receiving, or retrieving the three-dimensional model through various data acquisition methods. In some embodiments, the obtaining step may comprise generating the model in real-time, importing a previously created model, or accessing stored model data from a database or cloud storage system. In various embodiments, a 3D foot model such as 3D foot model 108 may be provided. In various embodiments, 3D foot model 108 may be generated in situ by one or more 3D scanners. Alternatively, the model may be created through photogrammetry techniques, structured light scanning, laser scanning, or computed tomography imaging. For example, and without limitation, one or more scanners, such as scanner 104 described above, may be utilized to form a 3D foot model 108. In certain other embodiments, one or more 3D scanners may be mounted on actuating arms configured to capture the geometric features and contours of a human foot. The capturing process may involve scanning, imaging, measuring, or digitizing the foot geometry through various sensing modalities. 3D foot model 108 may be a digital representation of the entirety of an individual's foot (such as foot 101) or a portion thereof. For example, and without limitation, 3D foot model 108 may be captured or provided that contains the geometrical and anatomical information of the sole of an individual's foot. The capturing step may alternatively involve recording, documenting, or mapping the foot geometry through direct contact methods, non-contact optical methods, or hybrid sensing approaches. In another example, 3D foot model 108 may be a digital representation of the entirety of an individual's foot, such as the sole, instep, outstep, bridge, ball, toes, arch, ankle, heel or portions of a human leg. In various embodiments 3D foot model 108 may be a digital representation of the internal anatomical features of human foot, including skeletal, musculature, ligamental, tendons, and vasculature, among others. For example, and without limitation, 3D foot model 108 may be used and reused in accordance with method 300. The utilization step may alternatively involve applying, implementing, employing, or leveraging the model data for subsequent processing steps.

With continued reference to FIG. 3, method 300 may include, at step 310, generating a three-dimensional model of an orthotic insole based on the 3D model of the individual's foot. Alternatively, step 310 may involve creating, constructing, building, developing, or synthesizing the three-dimensional insole model through computational design processes. The generating step may comprise calculating optimal insole geometry, deriving customized contours, or formulating patient-specific structural parameters. For example, and without limitation, the three-dimensional model of the orthotic insole may be the same or similar to insole model 116 as described above. In various embodiments, insole model 116 may be generated by one or more computing nodes or processors, such as processor 112 based on 3D foot model 108. The generation process may alternatively be performed through distributed computing systems, cloud-based processing platforms, or specialized design software applications. In various embodiments, generating the 3D insole model 116 may include analyzing 3D foot model 108 to determine areas requiring support or cushioning. The analyzing step may alternatively involve evaluating, assessing, examining, or investigating the foot model data to identify biomechanical requirements. This analysis may comprise processing geometric data, interpreting anatomical features, or computing load distribution patterns. In various embodiments, generating the 3D insole model 116 may include applying one or more biomechanical principles to design an insole shape that addresses the individual's specific needs. The applying step may alternatively involve implementing, utilizing, incorporating, or integrating biomechanical design rules through algorithmic processes, rule-based systems, or optimization techniques. In various embodiments, generating 3D insole model 116 may include incorporating one or more of a gait analysis, pressure mapping, geometrical or anatomical landmarks, or the like of an individual to design an insole customized for an individual. The incorporating step may alternatively involve integrating, combining, merging, or fusing multiple data sources through data fusion algorithms, weighted averaging methods, or multi-criteria optimization approaches. In certain embodiments, one or more supplemental data sets, such as the gait analysis, pressure mapping or geometric or anatomical landmarks may be provided to a system such as system 100 to design 3D insole model. The providing step may alternatively involve supplying, delivering, transmitting, or inputting the supplemental data through various communication protocols, data transfer methods, or user interface mechanisms. In certain embodiments, the one or more 3D scanners, such as scanner 104 may capture the gait analysis, pressure mapping or geometrical or anatomical landmarks to design 3D insole model 116. The capturing process may alternatively involve recording, measuring, monitoring, or tracking the biomechanical parameters through real-time sensing, periodic sampling, or continuous data acquisition methods.

With continued reference to FIG. 3, method 300 may include, at step 315, dividing the 3D insole model 116 into a plurality of zones, each zone having specified mechanical properties. Alternatively, step 315 may involve partitioning, segmenting, sectioning, or subdividing the insole model through automated algorithms, manual delineation, or hybrid segmentation approaches. The dividing process may comprise creating discrete regions, establishing functional areas, or defining property-specific domains within the insole geometry. In various embodiments, one or more computing nodes or processors, such as processor 112 may divide 3D insole model 116 into a plurality of zones 204, 208, 212 as shown in FIG. 2. The configuration step may alternatively involve programming, setting up, establishing, or initializing the processing systems to perform the zoning operations through software algorithms, machine learning models, or rule-based classification systems. In various embodiments, dividing the 3D insole model 116 may include identifying 3D insole model 116 into a plurality of zones 204, 208, 212 and a plurality of transition areas (such as 216 in FIG. 2) between adjacent zones. The identifying step may alternatively involve recognizing, detecting, locating, or determining the zone boundaries through image processing techniques, geometric analysis methods, or pattern recognition algorithms. This zoning step 315 may allow for precise customization of the 3D insole model 116 to address specific needs of different areas of the individual's foot corresponding to the plurality of zones. The customization process may alternatively involve tailoring, adapting, personalizing, or optimizing the insole design through parametric modeling, iterative refinement, or multi-objective optimization techniques. For example, and without limitation, a first zone corresponding to a high-pressure area may be designed with specified mechanical properties such as greater cushioning or support. The designing step may alternatively involve configuring, specifying, defining, or establishing the zone properties through material selection algorithms, property mapping functions, or biomechanical optimization processes. In another example, a second zone corresponding to a low-pressure area or area requiring more stability may be designed with specified mechanical properties such as greater stiffness. Zoning 315 may be based on geometric contours or anatomical landmarks of 3D foot model 108. The basing process may alternatively involve deriving from, referencing, utilizing, or grounding the zoning decisions on the foot model data through computational analysis, statistical modeling, or clinical decision support systems. In certain embodiments, zoning 315 may include analyzing pressure distribution data, biomechanical analysis, gait analysis or the like to identify the plurality of zones and the unique target mechanical properties associated with that zone. The analyzing step may alternatively involve processing, evaluating, interpreting, or computing the biomechanical data through signal processing algorithms, statistical analysis methods, or machine learning classification techniques. For example, and without limitation, the specified mechanical properties may be one or more of stiffness, flexibility, porosity, density or the like, each achievable by controlled variation of viscous thread coiling parameters during fabrication, to be described below. The achieving process may alternatively involve realizing, attaining, producing, or manifesting the desired properties through parameter optimization, process control, or material engineering approaches. In various embodiments, zoning 315 may enable fabrication of a highly personalized and customized physical orthotic insole with tailored support and comfort to different regions of the foot within a single, integrated structure. The enabling step may alternatively involve facilitating, allowing, permitting, or supporting the fabrication process through design optimization, manufacturing planning, or process parameter specification.

With continued reference to FIG. 3, method 300 may include, at step 320, generating printer instructions for fabricating an orthotic insole using viscous thread printing (VTP). Alternatively, step 320 may involve creating, formulating, developing, or producing the printer instructions through automated code generation, template-based programming, or custom algorithm development. The generating process may comprise compiling machine commands, encoding fabrication parameters, or translating design specifications into executable manufacturing instructions. Printer instructions may be the same or similar to printer instructions 120 as described above. In various embodiments, printer instructions 120 may produce the specified mechanical properties 133 in each zone (204, 208, 212, etc.) by controlling viscous thread coiling parameters 124 during deposition of said viscous threads. The controlling step may alternatively involve regulating, managing, adjusting, or modulating the coiling parameters through real-time feedback systems, predictive control algorithms, or adaptive parameter adjustment mechanisms. In certain embodiments, generating the printer instructions 120 may include generating a digital representation of the three-dimensional structure of 3D insole model, including the plurality of identified zones 204, 208, 212 formed by layers of viscous threads. The generating process may alternatively involve creating, constructing, building, or synthesizing the digital representation through computational modeling, geometric processing, or layer-by-layer decomposition algorithms. Further, generating the printer instructions 120 may include generating viscous thread coiling parameters 124 that will achieve the specified mechanical properties 133 in each zone (204, 208, 212) and transition area (216) during deposition of viscous threads. The achieving step may alternatively involve realizing, attaining, producing, or manifesting the desired properties through parameter optimization, inverse design methods, or predictive modeling approaches. In various embodiments, viscous thread coiling parameters 124 may include one or more of extrusion rate, print head translation speed, print height and thread diameter associated with a printing device such as printing device 136 described above. The association process may alternatively involve linking, connecting, correlating, or relating the parameters to the printing device through calibration procedures, system characterization, or performance mapping techniques. In various embodiments, viscous thread coiling parameters 124 may include ironing by the printing device 136. The ironing process may alternatively involve smoothing, flattening, compacting, or surface finishing through controlled contact pressure, temperature regulation, or mechanical processing techniques.

Even further, in various embodiments, generating printer instructions 124 may include applying continuous property gradients corresponding to transition areas (such as 216) between adjacent zones by gradually varying viscous thread coiling parameters 124 during continuous deposition of the orthotic insole 140. The applying step may alternatively involve implementing, establishing, creating, or introducing the property gradients through mathematical interpolation functions, spline-based transitions, or gradient descent optimization methods. The varying process may alternatively involve modulating, adjusting, changing, or transitioning the coiling parameters through parametric equations, smooth interpolation algorithms, or adaptive control strategies. In some cases, printer instructions 120 may incorporate gradual parameter variations to create smooth transitions between adjacent zones with different properties. The incorporating step may alternatively involve integrating, embedding, including, or building in the parameter variations through algorithmic programming, rule-based systems, or optimization-driven approaches. The resulting printer instructions 120 may guide the viscous thread printing device 136 in depositing material in a manner that produces the specified mechanical properties 133 in each zone through precise control of the viscous thread coiling parameters 124 during the fabrication process. The guiding process may alternatively involve directing, instructing, controlling, or steering the printing device through command sequences, motion planning algorithms, or real-time process control systems.

In various embodiments, method 300 may include determining viscous thread coiling parameters 124 via a predictive model (such as predictive model 128) to map viscous thread coiling parameters 124 to specified mechanical properties 133 at each zone and transition area of one or more thermoplastic materials. The determining step may alternatively involve calculating, computing, deriving, or establishing the coiling parameters through machine learning algorithms, regression analysis, neural network processing, or optimization techniques. The mapping process may alternatively involve correlating, relating, linking, or associating the parameters to properties through experimental validation, computational simulation, or hybrid modeling approaches. In various embodiments, predictive model 128 may determine, based on stored relationships between viscous thread coiling parameters 124 and resulting specified mechanical properties 133 for at least one material 134, VTP instructions 120 which will, upon deposition of viscous threads, affect the viscous thread coiling parameters 124 that will achieve the specified mechanical properties 133. The determining process may alternatively involve calculating, computing, deriving, or establishing the instructions through inverse modeling, optimization algorithms, or iterative refinement processes. Predictive model 128 may be formed by extensively mapping input parameters to output mechanical properties, wherein predictive model 128 may navigate the mechanical property space, granting the ability to apply inverse design to desired mechanical response. The forming process may alternatively involve creating, developing, building, or constructing the model through data collection, training procedures, or validation protocols. The navigating step may alternatively involve exploring, traversing, searching, or investigating the property space through multi-objective optimization, Pareto frontier analysis, or constraint satisfaction techniques.

Processor 112 and predictive model 128 may be communicatively coupled to database 132 that stores the relationships between viscous thread coiling parameters 124 and resulting specified mechanical properties 133 for the at least one material 134. The coupling process may alternatively involve connecting, linking, interfacing, or integrating the components through relational databases, NoSQL systems, distributed storage architectures, or cloud-based data repositories. The storing step may alternatively involve maintaining, retaining, preserving, or archiving the relationship data through various data management systems, backup protocols, or version control mechanisms. For example, and without limitation, VTP instructions 120 may specify motions and operational characteristics of printing device 136 to deposit coils of viscous threads having viscous thread coiling parameters 124 that effect the specified mechanical properties 133 for a given material 134 in each identified zone (204, 208, 212) and transition areas 216 with which the orthotic insole 140 is printed. The specifying step may alternatively involve defining, detailing, prescribing, or establishing the operational parameters through G-code commands, proprietary machine instructions, or standardized manufacturing protocols. The depositing process may alternatively involve extruding, placing, laying down, or applying the viscous threads through controlled extrusion, precision positioning, or layer-by-layer fabrication techniques. In various embodiments, the material 134 may be one or more thermoplastic materials suitable for viscous thread printing. Predictive model 128 may further gradually apply continuous property gradients between adjacent zones (204, 208, 212), such as in the transition areas 216, by gradually varying the viscous thread coiling parameters 124 in the printer instructions 120. The applying step may alternatively involve implementing, establishing, creating, or introducing the gradients through parametric equations, interpolation functions, or adaptive control algorithms. The varying process may alternatively involve modulating, adjusting, transitioning, or changing the parameters through smooth mathematical functions, spline-based interpolation, or gradient optimization techniques.

With continued reference to FIG. 3, method 300 may include, at step 325, printing the orthotic insole 140 based on the printer instructions 120. Alternatively, step 325 may involve fabricating, manufacturing, producing, or constructing the orthotic insole through additive manufacturing processes, layer-by-layer deposition, or continuous extrusion techniques. The printing process may comprise extruding material, depositing threads, building layers, or forming the three-dimensional structure through controlled material placement and thermal processing. In various embodiments, printing the orthotic insole 140 may be performed by a 3D printing device, such as a viscous thread printing device 136 described above. The performing step may alternatively involve executing, carrying out, implementing, or conducting the fabrication process through automated manufacturing systems, robotic control, or computer-guided production methods. Printing device 136 may print an orthotic insole 140 based on the 3D insole model 116, having specified mechanical properties 133, utilizing the VTP instructions 120. The configuring step may alternatively involve setting up, programming, calibrating, or preparing the printing device through software initialization, hardware calibration, or system optimization procedures. The utilizing process may alternatively involve employing, applying, implementing, or executing the instructions through command interpretation, motion control, or process parameter regulation. Printing device 136 may be a tabletop 3D printer. Printing device 136 may be one or more 3D printers operatively and communicatively coupled to one another to print orthotic insole 140 continuously, simultaneously or in stages. The coupling process may alternatively involve connecting, linking, networking, or integrating multiple printers through coordinated manufacturing protocols, distributed processing systems, or synchronized production workflows. The printing operation may alternatively involve manufacturing, fabricating, producing, or constructing the insole through sequential processing, parallel production, or hybrid manufacturing approaches.

In certain embodiments, such as shown in FIGS. 4A-4E, the present disclosure may provide for a customized orthotic insole 140 produced by a viscous thread printing process, such as method 300. In various embodiments, the orthotic insole 140 manufactured by a process 300 may include: obtaining a three-dimensional model of a user's foot; generating a corresponding insole geometry tailored to that model; partitioning the insole geometry into a plurality of zones each having specified target mechanical properties; translating those target properties into viscous thread printing instructions that prescribe zone-specific coiling parameters; and depositing viscous threads along toolpaths that implement the printing instructions so as to realize the specified properties in the printed structure.

Said method 300 imparts spatially varying mechanical properties (such as specified mechanical properties 133) throughout the three-dimensional monolithic structure of orthotic insole 140. The customized orthotic insole 140 may be defined, at least in part, by being fabricated through deposition of deformable thermoplastic threads into coil-based microstructures with controlled coiling parameters (e.g., viscous thread coiling parameters 124), where said coiling parameters are varied across the plurality of identified zones (204, 208, 212) to yield target mechanical responses of the orthotic insole 140, such as stiffness, cushioning, porosity, and durability. As described hereinabove, said zones may correspond to anatomical regions of an individual's foot, pressure distribution mapping, gait analysis, or biomechanical principles identified from the 3D foot model 108 or additional data streams. In certain embodiments, the orthotic insole 140 may also include a plurality of transition areas (such as 216) having continuous property gradient that avoid abrupt interfaces, seams, or the need for adhesives or jointing.

In some embodiments, the viscous thread printing process generates a continuous lattice or coil-based microstructure whose local coil density, coil diameter, coil pitch, and inter-coil connectivity are governed by the prescribed coiling parameters. The customized orthotic insole thereby includes a multi-zone internal architecture in which mechanical properties are engineered by adjusting, for each zone, at least one of: the ratio of extrusion rate to print head translation speed, the ratio of print height to thread diameter, and the spatial orientation or path curvature of deposited threads. Adjusting these inputs yields predictable changes in macro-scale stiffness and porosity at the point of deposition. In certain embodiments, a predictive model and/or material parameter map correlates input coiling parameters and toolpath features to resultant mechanical responses, enabling inverse design of the insole structure to meet zone-specific targets. The product-by-process insole includes continuous property gradients at interfaces between zones, implemented by gradually varying one or more coiling parameters across a boundary region, thereby blending local lattice parameters to avoid discrete material interfaces.

In exemplary embodiments, the manufacturing process that defines the product includes a computation step wherein the insole geometry is discretized into meshes corresponding to the zones, and a slicer generates printer-readable instructions that encode zone-specific coiling parameters and interface gradients. The printer executes the instructions to deposit viscous threads in situ, with feedback control available to maintain thread diameter and coil formation fidelity. The resulting product incorporates the prescribed internal architecture with a spatially graded property field that conforms to the user's anatomical and biomechanical requirements. Because property variation is achieved through process control rather than material substitution, the orthotic insole is seamless, integrated, and durable, with reduced risk of delamination or interface failure.

Orthotic insole 140 may be a customized orthotic insole configured for use by an individual based on the 3D foot model 108 and target mechanical properties in respective zones thereof. As shown in FIGS. 4A-4E, orthotic insole 140 may be a three-dimensional structure fabricated using viscous thread printing. The three-dimensional structure comprises multiple zones having different specified mechanical properties as described herein. The specified mechanical properties 133 may be produced in each zone by varying the viscous thread coiling parameters 124 during deposition of the viscous threads forming the three-dimensional structure. Orthotic insole 140 may include continuous property gradients between adjacent zones (e.g., in the transition zones 216), produced by gradually varying the viscous thread coiling parameters during fabrication. In various embodiments, specified mechanical properties 133 may include one or more of stiffness, density, porosity and flexibility, among others. In certain n embodiments, orthotic insole 140 may be fabricated from a single thermoplastic material, with distinct mechanical properties 133 in each zone 204, 208, 212 achieved solely through variation of the viscous thread coiling parameters 124.

The morphology of the product is further characterized by its zone layout relative to anatomy-derived features from the foot model. In some embodiments, the zones include at least a heel region, a medial longitudinal arch region, a lateral column region, a metatarsal head region, and a toe-off region, each having distinct target stiffness and porosity profiles derived from the user's pressure distribution. For example, a low-stiffness, higher-porosity cushion zone may be generated under peak pressure locations identified in the foot model, while a higher-stiffness support zone is generated along the medial arch to control pronation. The continuous gradients between these zones extend over predefined transition distances to maintain uniform feel and durability under cyclical loading. The resultant product exhibits an integrated, monolithic structure tuned to the user's biomechanics, without use of multi-material lamination or adhesives.

In certain embodiments, orthotic insole 140 may include high-density accumulation regions forming at least one of a wall (143 shown in FIGS. 4A-4E), base (144 shown in FIGS. 4A-4E), top surface (141 shown in FIGS. 4A-4E), edge (142 shown in FIGS. 4A-4E), or interstitial portion of the orthotic insole. Further, each accumulation region may be integrated via small gradients into the functional internal portions of the insole to form a solid, durable foundation and perimeter of the orthotic. These accumulation regions may be deposited by locally increasing coil density and/or effective material accumulation through parameter selection and toolpath dwell, thereby enhancing durability, edge stability, and outsole coupling while maintaining functional internal cushioning and support. The accumulation regions may be integrated into adjacent functional zones via small gradients that connect high-density structures to lower-density interior structures.

Moreover, orthotic insole 140 may include a top surface layer 141. Top surface layer 141 may be applied after fabrication by viscous thread printing or formed continuously and integrally by viscous thread printing. In various embodiments, top surface layer 141 may be formed by additions or subtractions performed by the viscous thread printing device 136. For example, and without limitation, top surface layer 141 may be formed in-process by ironing the topmost layer of viscous threads through contact with the relatively warm extruder head, smoothing and flattening the topmost layer of viscous threads. The ironing process may involve controlling the temperature of the extruder head to maintain optimal thermal conditions for surface finishing while avoiding degradation of the underlying thread structure. The contact pressure and translation speed of the extruder head during ironing may be precisely controlled to achieve uniform surface texture and thickness across the top surface layer 141.

In another example, a distinct and separate top surface layer 141 may be applied to orthotic insole 140 after fabrication. In said example, deposited viscous threads of the orthotic insole 140 may include inherently formed loops, which facilitate coupling with an external top surface layer 141 having corresponding hooks. These inherently formed loops may result from the controlled coiling parameters during the viscous thread printing process, where specific combinations of extrusion rate, print head translation speed, and thread diameter create loop structures that extend above the general surface plane of the insole. The loop structures may be strategically positioned across predetermined areas of the top surface to provide optimal engagement with the hook-and-loop fastening system. The distinct top surface layer 141 may be formed from an EVA foam and be configured for rapid removability and replaceability. The EVA foam material may be selected for its cushioning properties, moisture resistance, and compatibility with the hook-and-loop attachment mechanism. The removable nature of the top surface layer 141 allows for easy cleaning, replacement due to wear, or substitution with alternative surface materials having different tactile properties or therapeutic characteristics. The replaceability feature enables customization of the user experience without requiring fabrication of an entirely new orthotic insole, thereby extending the useful life of the underlying printed structure and providing cost-effective maintenance options for end users.

In some embodiments, the product includes features that facilitate clinical adjustment and refurbishment throughout the orthotic's service life. The printed lattice structure exposes surface loops and microstructures that can engage with supplemental covers, pads, or localized inserts without requiring adhesives or permanent bonding agents, allowing clinicians to modify top-layer feel, redistribute pressure patterns, or add therapeutic elements while preserving the underlying tuned lattice architecture. These surface features may include strategically positioned loop formations created through controlled viscous thread coiling parameters that naturally extend above the general surface plane, providing mechanical attachment points for hook-and-loop fastening systems or snap-fit accessories. The accumulation regions at the base and perimeter provide a robust foundation for repeated attachment and removal of such accessories, featuring enhanced material density and structural integrity that can withstand cyclical loading and unloading forces associated with accessory installation and removal. This design approach allows the customized orthotic insole to be adapted over time to evolving patient needs, accommodating changes in foot morphology, gait patterns, or therapeutic requirements without necessitating complete replacement of the underlying printed structure. The modular accessory system enables cost-effective maintenance and customization, extending the useful life of the primary orthotic structure while providing flexibility for clinical adjustments based on patient feedback or changing biomechanical requirements.

In certain embodiments, orthotic insole 140 may be formed from a single thermoplastic material and fabricated continuously on a single printing device, such as printing device 136 described herein, eliminating the need for multi-material systems, adhesive bonding, or post-processing assembly operations. The regional differences in mechanical properties in corresponding zones and transition areas may be achieved solely through local modulation of viscous thread coiling parameters 124, including, without limitation, modulation of extrusion rate, print head translation speed, print height, and thread diameter, among others. This single-material approach provides several advantages including consistent material compatibility throughout the structure, elimination of potential delamination at material interfaces, simplified manufacturing logistics, and reduced material inventory requirements. The continuous fabrication process ensures seamless integration between zones with different mechanical properties, as the same thermoplastic material is deposited with varying coiling parameters to achieve the desired local characteristics. The printing device 136 may dynamically adjust these parameters in real-time during the fabrication process, transitioning smoothly between different coiling configurations as the print head moves across zone boundaries, thereby creating the continuous property gradients that characterize the transition areas between functional zones.

In various embodiments, the product-by-process insole is produced from a single thermoplastic material selected for compatibility with viscous thread deposition and designed to provide long-term resilience under repeated compressive and shear loads encountered in typical footwear applications. The material selection criteria may include optimal viscosity characteristics at extrusion temperatures, appropriate glass transition temperature for room temperature flexibility, resistance to fatigue under cyclical loading, and biocompatibility for extended skin contact. By virtue of the controlled coiling microstructure, the insole demonstrates breathability through interconnected porosity created by the spaces between adjacent coils, energy return modulated by coil geometry and elastic properties of the coil structure, and directional stiffness engineered by toolpath orientation and coil alignment patterns. The interconnected porosity allows for air circulation and moisture management, while the coil geometry can be tuned to provide specific energy return characteristics that enhance walking efficiency or running performance. The process further enables the creation of anisotropic regions in the insole, where stiffness in the medial-lateral direction differs from the anterior-posterior direction, achieved by aligning coil paths and adjusting local parameter ratios to create preferential load-bearing directions that support natural foot biomechanics. These anisotropic properties can be particularly beneficial in addressing specific gait abnormalities or providing targeted support for athletic applications. The printed structure may be configured to withstand routine wear, moisture exposure, and temperature variations encountered in footwear use through appropriate material selection and coiling parameter optimization that maintains structural integrity across expected environmental conditions. The high-density perimeter or base regions may be specifically tuned to accommodate bonding to a shoe midsole through mechanical interlocking features or adhesive compatibility, or to provide standalone durability when used as a removable insert that must withstand repeated insertion and removal cycles without structural degradation or loss of therapeutic effectiveness.

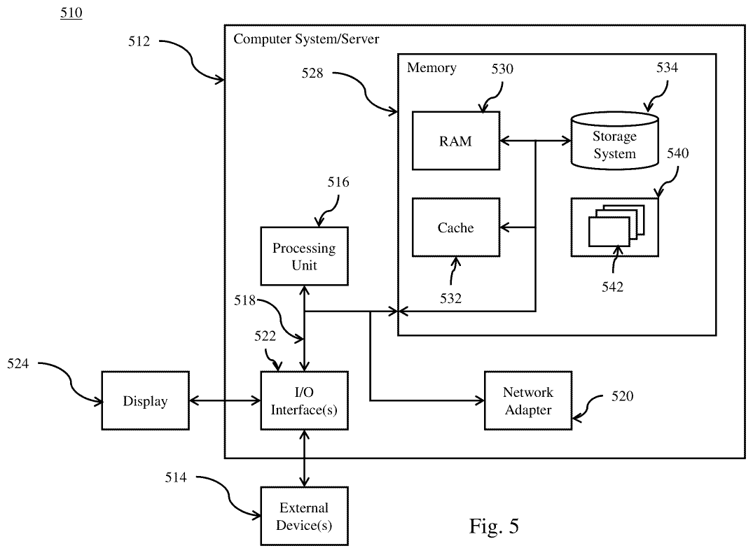

Any method step or component of the described system may be implemented automatedly through one or more computing nodes, processors, or controllers. Referring now to FIG. 5, a schematic of an example of a computing node is shown. Computing node 510 is only one example of a suitable computing node and is not intended to suggest any limitation as to the scope of use or functionality of embodiments described herein. Regardless, computing node 510 is capable of being implemented and/or performing any of the functionality set forth hereinabove.

In computing node 510 there is a computer system/server 512, which is operational with numerous other general purpose or special purpose computing system environments or configurations. Examples of well-known computing systems, environments, and/or configurations that may be suitable for use with computer system/server 512 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 512 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 512 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 512 in computing node 510 is shown in the form of a general-purpose computing device. The components of computer system/server 512 may include, but are not limited to, one or more processors or processing units 516, a system memory 528, and a bus 518 that couples various system components including system memory 528 to processor 516.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (Pie), and Advanced Microcontroller Bus Architecture (AMBA).

Computer system/server 512 typically includes a variety of computer system readable media. Such media may be any available media that is accessible by computer system/server 512, and it includes both volatile and non-volatile media, removable and non-removable media.

System memory 528 can include computer system readable media in the form of volatile memory, such as random access memory (RAM) 530 and/or cache memory 532. Computer system/server 512 may further include other removable/non-removable, volatile/non-volatile computer system storage media. By way of example only, storage system 534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 518 by one or more data media interfaces. As will be further depicted and described below, memory 528 may include at least one program product having a set (e.g., at least one) of program modules that are configured to carry out the functions of embodiments of the disclosure.

Program/utility 540, having a set (at least one) of program modules 542, may be stored in memory 528 by way of example, and not limitation, as well as an operating system, one or more application programs, other program modules, and program data. Each of the operating system, one or more application programs, other program modules, and program data or some combination thereof, may include an implementation of a networking environment. Program modules 542 generally carry out the functions and/or methodologies of embodiments as described herein.

Computer system/server 512 may also communicate with one or more external devices 514 such as a keyboard, a pointing device, a display 524, etc.; one or more devices that enable a user to interact with computer system/server 512; and/or any devices (e.g., network card, modem, etc.) that enable computer system/server 512 to communicate with one or more other computing devices. Such communication can occur via Input/Output (I/O) interfaces 522. Still yet, computer system/server 512 can communicate with one or more networks such as a local area network (LAN), a general wide area network (WAN), and/or a public network (e.g., the Internet) via network adapter 520. As depicted, network adapter 520 communicates with the other components of computer system/server 512 via bus 518. It should be understood that although not shown, other hardware and/or software components could be used in conjunction with computer system/server 512. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for manufacturing a customized orthotic insole, the method comprising:
obtaining a three-dimensional model of an individual's foot;
generating a three-dimensional model of an orthotic insole based on the three-dimensional model of the individual's foot;
dividing the three-dimensional model of the orthotic insole into multiple zones, each zone having specified mechanical properties;
generating printer instructions for fabricating the orthotic insole using viscous thread printing, wherein the printer instructions are configured to produce the specified mechanical properties in each zone by controlling viscous thread coiling parameters during deposition of viscous threads; and
printing an orthotic insole based on the printer instructions.

2. The method of claim 1, wherein obtaining a three-dimensional model of the individual's foot comprises capturing a shape and a plurality of contours of the individual's foot with a three-dimensional scanner.

3. The method of claim 1, wherein generating the three-dimensional model of the orthotic insole further comprises analyzing at least one of an anatomical landmark, a gait analysis or a pressure map of the individual.

4. The method of claim 1, wherein the specified mechanical property comprises at least one of stiffness, flexibility or porosity.

5. The method of claim 1, wherein dividing the three-dimensional model of the orthotic insole into multiple zones comprises identifying transition areas between the multiple zones.

6. The method of claim 5, generating printing instructions comprises:
generating a digital representation of the orthotic insole's three-dimensional structure including the multiple zones; and
generating viscous thread coiling parameters based on the specified mechanical properties of each zone and transition areas of the orthotic insole.

7. The method of claim 6, wherein the printer instructions comprises at least one of extrusion rate, print head translation speed, print height, and thread diameter.

8. The method of claim 6, wherein generating the printing instructions comprises applying continuous property gradients corresponding to the transition areas between identified zones by gradually varying the viscous thread coiling parameters.

9. The method of claim 6, further comprising determining viscous thread coiling parameters via a predictive model configured to map the viscous thread coiling parameters to the specified mechanical properties at each identified zone and transition area.

10. A system for manufacturing a customized orthotic insole, the system comprising:
a three-dimensional scanner configured to obtain a three-dimensional model of an individual's foot;
a processor configured to:
generate a three-dimensional model of an orthotic insole based on the three-dimensional model of the individual's foot;
divide the three-dimensional model of an orthotic insole into a plurality of zones, each zone having specified mechanical properties; and
generate viscous thread printing instructions including a plurality of viscous thread coiling parameters to print an orthotic insole based on the three-dimensional model of the orthotic insole and the specified mechanical properties of each zone;
a viscous thread printing device configured to deposit viscous threads based on the viscous thread printing instructions.

11. The system of claim 10, wherein the plurality of viscous thread coiling parameters comprises at least one of extrusion rate, print head translation speed, print height and thread diameter.

12. The system of claim 10, further comprising a material database storing relationships between viscous thread coiling parameters and specified mechanical properties for at least one thermoplastic material.

13. The system of claim 12, wherein the processor is configured to use a predictive model to determine viscous thread coiling parameters that will produce the specified mechanical properties in each zone.

14. The system of claim 10, wherein the processor is further configured to apply continuous property gradients between adjacent zones by gradually varying the viscous thread coiling parameters in the printing instructions.

15. A customized orthotic insole produced by a process comprising the steps of:
obtaining a three-dimensional model of an individual's foot;
generating a three-dimensional model of an orthotic insole based on the three-dimensional model of the individual's foot;
dividing the three-dimensional model of the orthotic insole into multiple zones, each zone having specified mechanical properties;
dividing the three-dimensional model of the orthotic insole into at least one transition area between adjacent zones;
generating printer instructions for fabricating the orthotic insole using viscous thread printing, the printer instructions are configured to produce the specified mechanical properties in each zone and transition area by controlling viscous thread coiling parameters during deposition of viscous threads; and
printing the customized orthotic insole via a viscous thread printing device based on the printer instructions, the customized orthotic insole comprising the plurality of viscous threads deposited in layers to form a three-dimensional structure the specified mechanical properties in a corresponding one of the multiple zones and transition area.

16. The customized orthotic insole of claim 15, wherein the customized orthotic insole is fabricated from a single thermoplastic material.

17. The customized orthotic insole of claim 15, wherein each of specified mechanical properties corresponding to each zone and transition area is achieved through variation of the viscous thread coiling parameters.

18. The customized orthotic insole of claim 15, further comprising a high-density accumulation region of viscous threads forming at least one of a wall and a base of the orthotic insole.

19. The customized orthotic insole of claim 15, wherein a top surface of the orthotic insole is ironed by the viscous thread printer during the printing.

20. The customized orthotic insole of claim 15, wherein a top surface of the orthotic insole is printed with a plurality of loops formed by the viscous threads, the loops configured to receive and engage with a replaceable top surface layer having a plurality of hooks.
